(19) United States
(12) Patent Application Publication
(10) Pub. No.: US 2014/0044425 A1
(43) Pub. Date: Feb. 13, 2014

(54) Dual mode fragrance-emitting apparatus and methods for improved safety during use

(71) Applicant: Brain Beesley, Draper, UT (US)

(72) Inventor: Brain Beesley, Draper, UT (US)

(21) Appl. No.: 13/507,932

(22) Filed: Aug. 8, 2012

(51) Int. Cl. A61L 9/03

(52) U.S. Cl. 392/386

(57) ABSTRACT

A presently preferred apparatus, and related methods are disclosed by which fragrance can be safely released from either a container melted candle or containerized wax particles using flameless heat conductively applied to the candle container or radiantly applied to the wax particle container across an air space.

### FIELD OF INVENTION

In general the present invention relates to emission of fragrances or aromas and more particularly to highly safe apparatus and methods for dual mode release of fragrance or aroma from either a candle or particles of wax.

### BACKGROUND

In large measure, the burning of candles to emit an aroma or fragrance into the air within a home or other building has been replaced by either melting a candle with an electrically actuated flat heating element to melt the candle and release the aroma or, using an entirely different apparatus, placing wax chips or particles in a flat bottom open top container followed by placing the entire flat bottom of the container on an electrically activated flat heating element. The second approach has raised safety concerns.

### BRIEF SUMMARY AND OBJECTS OF THE INVENTION

In brief summary, the present invention provides a single apparatus, and related methods, which can be used safely, without combustion and related pollution, in either of two ways to release fragrance by conductively melting a candle or by radiantly melting wax chips placed in a container which is largely spaced from a source of heat.

In view of the foregoing, it is a primary object of the present invention to provide improved, dual-use candle and chip melting and fragrance-emitting apparatus and methods, which provide for improved safety.

Another object is the provision of fragrance-emitting apparatus and methods, where the bottom of a wax-receiving container is predominantly spaced from the top surface of an associated heating element, by which significantly improved safety is achieved.

These and other objects and features of the present invention will be apparent from the following detailed description taken with reference to the accompanying drawings.

### BRIEF DESCRIPTION OF THE DRAWINGS

FIG. 1 is an exploded perspective of one presently preferred apparatus for practicing the present invention;

FIG. 2 is a vertical cross section of one portion of the apparatus of FIG. 1, being used to emit fragrance from a containerized melted candle; and

FIG. 3 is a vertical cross section showing utilization of both elements of the apparatus of FIG. 1 to melt and cause release of fragrance from wax chips.

### DETAILED DESCRIPTION OF THE ILLUSTRATED EMBODIMENT

Reference is now made specifically to the drawings wherein a presently preferred apparatus, generally designated 10, is illustrated. Apparatus 10 comprises a heating element, generally designated 12, which comprises a heating plate 14. Plate 14 comprises a top horizontally flat exposed surface 16. The housing 18 of the heating element 12 may comprise a ceramic material and may or may not comprise an exterior decorative design. Beneath the heating plate 14 is a resistor-based circuitry, of conventional construction, to which electricity is delivered through cord 20. Switch 22, on an electrical plug 24 may be conductively inserted into a household socket where 110 volt electricity is available.

The heating element 12, at the top of housing 18 is illustrated as comprising a rounded, upwardly-directed annular lip, which serves a purpose yet to be described. The base of the housing 18 of the heating element 12 also comprises an annular, downwardly-directed lip 28.

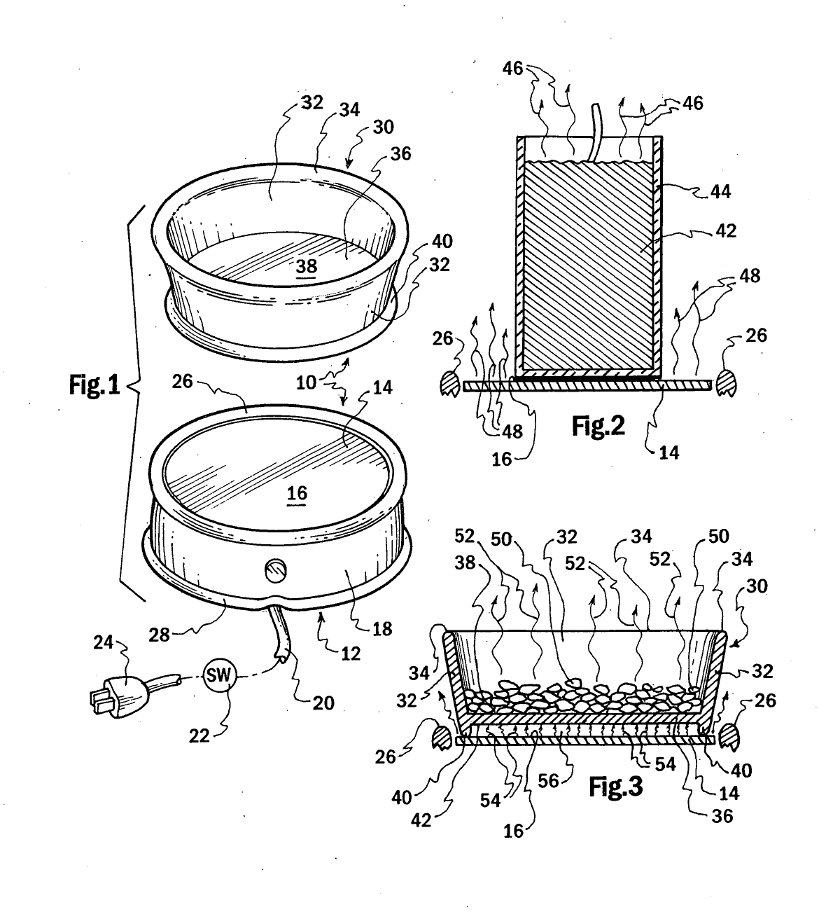

The apparatus 10 of FIG. 1 further comprises a ceramic container, generally designated 30. Container 30 may have a decorative exterior, if desired. Container 30 has an annular tapered side wall 32, shown as being essentially of uniform thickness and terminating in an annular upwardly-extending rounded edge 34. The container 30 comprises a bottom wall 36 consisting of a top flat surface 38, upon which wax particles or chips may be placed for purposes yet to be explained. The container 30 is open at its top and closed by bottom wall 36, with an annular downwardly directed lower lip or ring 40 purposefully extending downwardly below the bottom surface 42 (FIG. 3) for a purpose yet to be explained.

The open top container 30 is manually portable, so that it can be optionally manually placed away from the heating element 12 or can be stacked, at lip or ring 40 on the top of flat surface 16, the ring 40 having a diameter slightly less than the diameter of the flat surface 16 to accommodated such stacking.

When the user desires, the container 30 may be manually set to one side, the heating element 12 typically set on a flat surface, such as a table or a desk, the plug 24 inserted into a household wall socket, the switch 22 placed in an on position, with a containerized candle contiguously superimposed upon the top surface 16 of the heating plate 14, as shown in FIG. 2. Thus, the candle material 42 is melted by conduction within its container 44 to emit fragrance 46 flamelessly into the ambient air within the immediate location of the candle. Heat from the heating plate 14 is designated by the numeral 48, in FIG. 2. When the user no longer wishes fragrance from the candle, the plug 24 may be removed from the wall socket and/or the switch 22 may be closed manually, after which the remaining candle material 42 in container 44 will solidify and, thereafter, be available to be heated at desired intervals.

Reference is now made to FIG. 3. When the user desires fragrance 52 from wax particles or chips 50, wax chips or particles, in solid form, are placed through the open top of the container 30 so as to rest by force of gravity against the top surface 38 of the bottom wall 36 of container 30. The container 30 is placed on the top surface 16 of the heating plate 14. The electrical plug 24 is placed in a wall socket, with the switch 22 on, causing the heating plate 14 to generate heat, illustrated in FIG. 3 by the numerals 54. The ceramic container 30 at lower lip or ring 40 contiguously and annularly engages the top surface 16, but transfer of heat by conduction across ring 40 into the container 30 is very limited. Instead, by convection, the heat elevating from the top surface 16 of the heating plate 14 crosses the hollow space 56, contained within the annular lip 40 to radiantly heat and melt the wax particles or chips 50 within the container 30, whereby fragrance 52 emanating from the melted wax particles elevates into the ambient air surrounding the container 30.

When aroma from the wax particles is no longer desired, the plug 24 may be removed from the wall socket and/or the switch 22 may be manually closed so that electricity ceases to heat the heating plate 14. The material comprising the melted wax particles then solidifies within the container 30 for future use, when heated, to cause fragrance once more to infiltrate the ambient air adjacent to the containers.

The invention may be embodied in other specific forms without departing from the spirit of the essential characteristics thereof The present embodiments, therefore, are to be considered in all respects as illustrative and are not restrictive, the scope of the invention being indicated by the appended claims rather than by the foregoing description, and all changes which come within the meaning and range of equivalency of the claims are therefore intended to be embraced therein. What is claimed and described to be secured by Letters Patent is:

1-4. (canceled)

5. A dual mode apparatus of improved safety for releasing fragrance from candle material, without combustion, and from small particles, without combustion, the apparatus comprising:
an electrically-powered heating element comprising a flat top heating surface generating a predetermined temperature;
a gravity-retained but removable open-top container comprising a bottom wall including a bottom surface adapted to be manually placed above the heating surface, the container being contiguously supported by the heating plate only along a limited contact peripheral surface-engagement site, the bottom wall of the container merging with a peripheral skirt terminating in said peripheral surface, a concealed air space existing below the bottom surface and interior of the skirt;
whereby, with the removable container positioned as stated above, the predetermined temperature of the heating surface is convectionally communicated only in part across the air space thereby heating the bottom wall of the container to substantially lower safe temperature such that relatively small fragrance-emitting particles placed in the container safely melt but do not overheat;
a relatively large amount of candle material enclosed within an encasement, the encasement comprising a bottom wall having top and bottom surfaces, the top surface of a bottom wall of the encasement being placed in contiguous contact with the candle material;
whereby, with the open top container removed from the heating surface, and the bottom surface of the bottom wall of the candle material encasement contiguous with the heating surface, the predetermined temperature of the heating surface is substantially communicated by conduction directly to the bottom wall of the encasement such that the candle material is melted to a safe temperature which is substantially greater than temperature imposed on the fragrance-emitting particles.

6. A dual mode apparatus according to claim 5 wherein the skirt and the edge are annular and the heating surface circular in their respective configurations.

7. An apparatus for flamelessly emitting a fragrance in two modes comprising:
an electrically-powered heating element for reaching a predetermined temperature;
a first portable container having a large amount of candle material therein;
the heating element accommodating gravity placement of the first container directly and contiguously thereon to conductively and safely melt the candle material thereby releasing candle fragrance at a relatively high temperature derived from the first predetermined temperature of the heating element;
a second portable container having an opening at the top thereof for receiving small particles of fragrance-emitting material, the second container comprising a bottom wall in combination with downwardly extending a peripheral line contact stand defining a concealed hollow recess, the line contact of the stand removably and contiguously engaging the heating element when the second container is manually placed upon the heating element in the absence of the first container to safely and radiantly melt the wax particles by convection across the recess at another temperature, substantially lower than the relatively high one temperature, derived from the constant predetermined temperature of the heating element to thereby release wax particle fragrance.

8. A dual monitor, single temperature source method by which a user may safely cause release of fragrance by melting solid candle material comprising a relatively large mass and by melting relatively small wax particles, comprising the acts of:
placing a containerized relatively large quantity of scented candle material contiguously on a top surface of a heating element;
activating the heating element to a predetermined temperature to flamelessly conductively melt the candle material at a relatively high heat but safe temperature thereby releasing candle scent;
placing a container having a bottom and an opening through which relatively small scented particles have been placed in the container on the heating element so that only a limited area of the container is contiguous with the heating element and a relatively large space exists between the top surface of the heating element and the bottom of the container;
activating the heating element to the predetermined temperature to flamelessly and radiantly melt the wax particles across the large space at a temperature materially lower than the high temperature to release the scent of the particles.

9. A method by which a user may safely release fragrance flameleslly from melted scented particles, comprising the acts of:
placing a container, having a bottom and an opening through which fragrance-emitting particles are placed in the container, on the heating element so that only a small portion of the area of the bottom of the container is contiguous with the heating element and a relatively large concealed space exists between the heating element and the bottom of the container;
activating the heating element to a temperature which could be potentially hazardous if applied directly to the particles to flamelessly and radiantly melt the particles across the large space to thereby melt the wax particles at a safe lower temperature and safely release fragrance therefrom without monitoring and without changing the temperature of the heating element.
